(19) United States
(12) Patent Application Publication
(10) Pub. No.: US 2006/0089558 A1
(43) Pub. Date: Apr. 27, 2006

(54) Physiological parameter monitoring and data collection system and method

(75) Inventors: Kenneth Brakeley Welles II, Scotia, NY (US); John Erik Hershey, Ballston Lake, NY (US); Glenn Alan Forman, Niskayuna, NY (US); Jeffrey Michael Ashe, Gloversville, NY (US); Richard Louis Zinser JR., Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/973,599

(22) Filed: Oct. 27, 2004

(51) Int. Cl. A61B 5/04

(52) U.S. Cl. 600/509; 600/544; 600/511

(57) ABSTRACT

A physiological parameter monitoring system and method is described. The system includes a plurality of sensors configured to obtain from a subject at least one observable voltage containing two or more signals. The system also includes a data manager and a data auxiliary device. The data manager is in communication with the plurality of sensors and is configured to assemble and format data obtained by the plurality of sensors. The data manager is configured to isolate one of the two or more signals. The method includes placing a plurality of sensors in communication with a subject, transmitting data from the plurality of sensors to a data manager, and isolating a desired voltage signal from the plurality of voltage signals.

### BACKGROUND

The invention relates generally to a system and method for obtaining physiological measurements, and more particularly to a system and method for monitoring and collecting data on physiological parameters with a decreased noise level.

There are numerous instances where a need arises to monitor selected physiological parameters of both immobile and ambulatory subjects. The parameters most likely in need of monitoring include, but are not limited to, the subject's electrocardiogram (ECG), electroencephalogram (EEG), and heart rate. Further, the parameters may include an ECG and a heart rate of a fetus within the subject.

A significant challenge in obtaining good data is noise artifacts. Noise artifacts may be introduced through a variety of mechanisms. For example, noise may be introduced through competing signals, such as a mother's ECG and a fetus' ECG. Also, the activation of the subject's muscles may introduce noise or a motion-induced artifact. Further, a phenomenon called skin stretch, which reduces the local magnitude of the skin potential, produces a motion-induced artifact in voltage.

Many noise artifacts may be removed through signal processing techniques. For example, adaptive signal processing has been reported to mitigate noise by using voltage pickup leads positioned near muscles that are producing the interference and adaptively subtracting the interfering signal from the corrupted sought after signal. See, Luo, S. and Tompkins, W., Experimental Study: Brachial Motion Artifact Reduction in the ECG, Computers and Cardiology, p. 33-36 (1995). Additionally, a method for reducing motion-induced artifacts in voltage by incorporating a deformation gauge with a skin contact electrode to provide a reference signal for skin stretch noise estimation and subtraction is described in U.S. Pat. No. 5,978,693. That same patent also describes a skin-mounted physiological recording electrode assembly with a foam pad that is skin compliant. Further, the challenge of signal processing separation of a fetus ECG from its mother's ECG using a plurality of reference inputs has been described in Zarzoso, V. and Nandei, A., Noninvasive Fetal Electrocardiogram Extraction: Blind Separation Versus Adaptive Noise Cancellation, IEEE Transactions on Biomedical Engineering, Vol. 48, No. 1, p. 12-18 (January 2001).

Many known techniques for reducing noise in the voltages in the human body are based upon an assumption that the human body can be electrically modeled linearly. Specifically, the noise reduction techniques are based upon the assumption that observable voltages p measured by an electrode attached to a human body, arrayed as a p×1 column vector y, are related to directly unobservable source voltages q in the body, represented by a q×1 column vector x. The observable voltages p and the unobservable source voltages q may be represented in a mixing matrix M:

$$M = \begin{matrix}{e}_{11} & {e}_{12} & {e}_{13} & ⋯ & {e}_{1 q} \\ {e}_{21} & {e}_{22} & {e}_{23} & ⋯ & {e}_{2 q} \\ ⋯ & ⋯ & ⋯ & ⋯ & ⋯ \\ {e}_{p 1} & {e}_{p 2} & {e}_{p 3} & ⋯ & {e}_{pq}\end{matrix}$$

where y=Mx. Given that this model is linear, it exhibits many linear mathematical properties. The matrix M is dynamic and may change when the body changes position or conductivity changes, due to sweating, for example.

There exists a need for an efficacious methodology for gathering physiological data devoid of noise artifacts that render such physiological data suspect.

### BRIEF DESCRIPTION OF THE DRAWINGS

FIG. 1 illustrates a physiological data monitoring constructed in accordance with an embodiment of the invention.

FIG. 2 illustrates a physiological data monitoring constructed in accordance with another embodiment of the invention.

FIG. 3 schematically illustrates a simplified linear electrical model of the human body.

FIG. 4 illustrates the observable voltages of V₁ and V₂ of FIG. 3.

FIG. 5 illustrates the isolation of the heart signal V_{H} from the noise signal V_{N} of FIG. 3.

FIG. 6 illustrates process steps for isolating an electrical signal in accordance with another embodiment of the invention.

### SUMMARY

The present invention describes a system and a method for obtaining physiological measurements from which noise has been isolated.

One exemplary embodiment of the invention is physiological parameter monitoring system. The system includes a plurality of sensors configured to obtain from a subject at least one observable voltage containing two or more signals, a data manager in communication with the plurality of sensors and being configured to assemble and format data obtained by the plurality of sensors, and a data auxiliary device configured to receive the assembled and formatted data from the data manager. The data manager is configured to isolate one of the two or more signals.

One aspect of the physiological parameter monitoring system is that the at least one observable voltage is a potential difference between two of said plurality of sensors, and that the data manager is configured to isolate one of the two or more signals by manipulating mesh equations that model the physiological data of the two of said plurality of sensors.

Another exemplary embodiment of the invention is a method for collecting and monitoring physiological parameters. The method includes the steps of placing a plurality of sensors in communication with a subject, transmitting data from the plurality of sensors to a data manager, and isolating a desired voltage signal from the plurality of voltage signals. The plurality of sensors is configured to obtain a plurality of voltage signals from the subject. Further, the data manager isolates the desired voltage signal through a manipulation of mesh equations that model the physiological data of at least two of the plurality of sensors.

One aspect of the method for collecting and monitoring physiological parameters is that each of the plurality of voltage signals is a potential difference between two of the plurality of sensors, and the manipulation of mesh equations is accomplished through the use of resistive circuit analytical techniques.

These and other advantages and features will be more readily understood from the following detailed description of preferred embodiments of the invention that is provided in connection with the accompanying drawings.

### DETAILED DESCRIPTION OF EXEMPLARY EMBODIMENTS

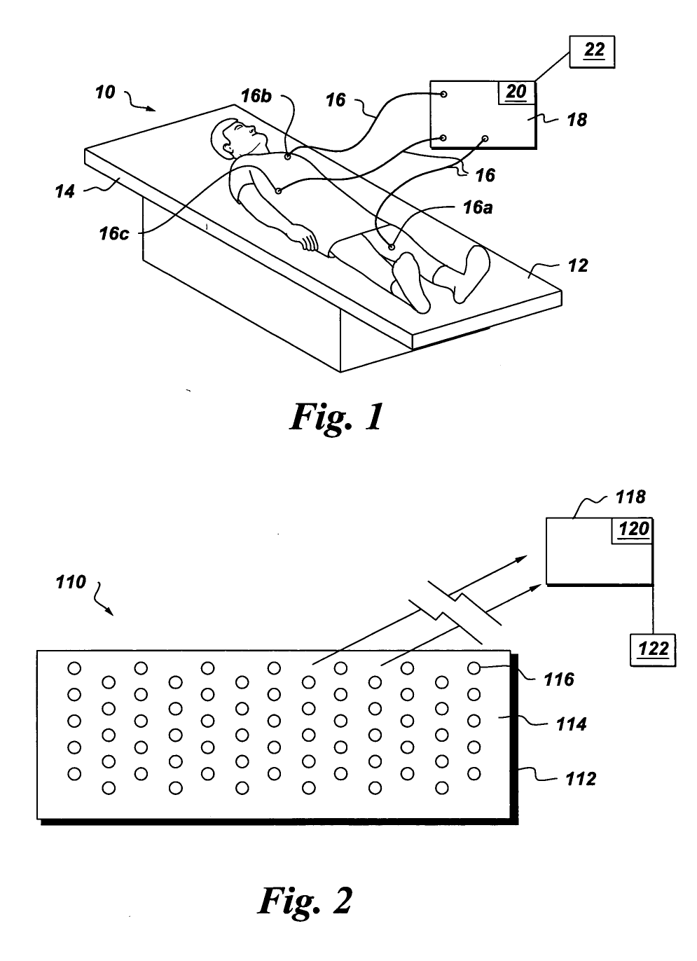

With reference to FIG. 1, there is depicted a physiological parameter monitoring system 10 that includes a table 12 or other structure upon which a subject may repose, a plurality of sensors 16 for obtaining physiological data from the subject, and a computational device 18. The sensors 16 are placed in contact with a subject's body and are each in communication with the computational device 18. The sensors 16 may be any type of sensor capable of obtaining physiological data from the subject, such as, for example, capacitive sensors, single electrode sensors, and Laplacian electrode sensors.

The computational device 18 includes a data manager 20 that is configured to assemble and format data received from the sensors 16. The computational device 18 further includes a data auxiliary device 22 configured to receive the assembled and formatted data from the data manager 20. The sensors 16 are shown in a particular position relative to the subject's body, but it should be appreciated that such position is merely representative and that any positioning of the sensors 16 that is suitable for obtaining a voltage signal is acceptable.

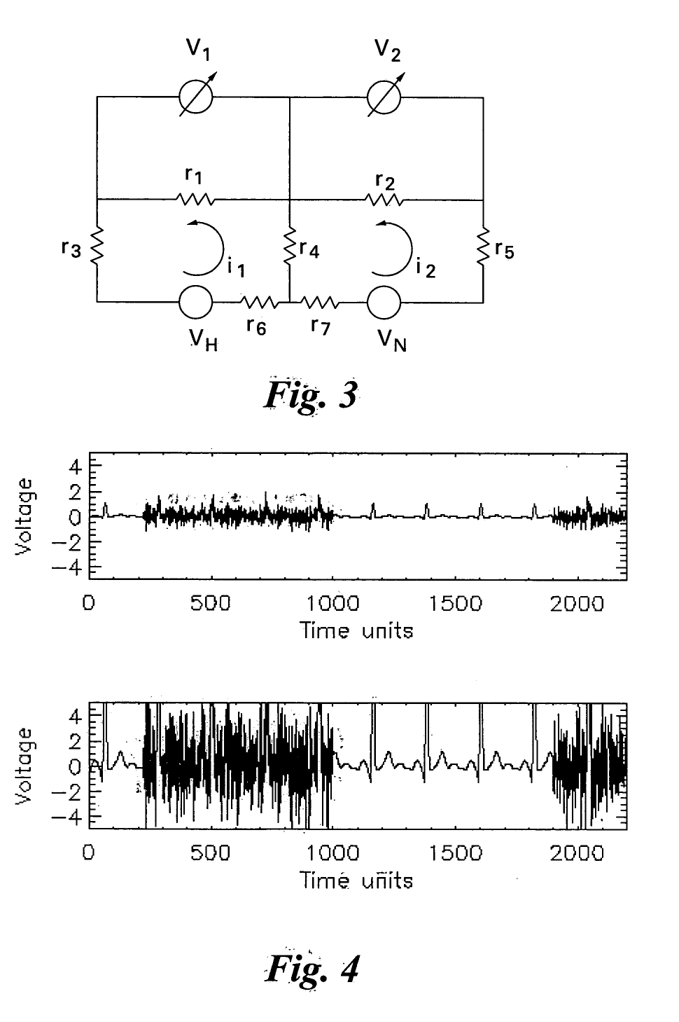

Referring now to FIG. 3, there is illustrated a simple linear electrical model of a human body. The model includes a pair of observable voltages V₁ and V₂, as well as two time-varying electrical voltage signals V_{H} and V_{N}. The voltage V₁ is the observable voltage across resistor r₁, while the voltage V₂ is the observable voltage across resistor r₂. The observable voltages V₁ and V₂ are in reality potential differences obtained through a pair of sensors 16. For example, observable voltage V₁ may be a potential difference between sensor 16a and sensor 16b (FIG. 1), while observable voltage V₂ may be a potential difference between sensor 16a and sensor 16c. The electrical voltage signal V_{H} corresponds to the voltage signal of the subject's heart, while the electrical voltage signal V_{N} corresponds to an interfering artifact or noise signal. The remaining resistances r₃, r₄, r₅, r₆, and r₇ correspond to additional resistive components to currents i₁ and i₂. Resistance r₄ is shared between the currents i₁ and i₂. Two mesh equations are derived from the model, namely
V₁=i₁×r₁
and
V₂=i₂×r₂.

FIG. 4 illustrates that there are two distinct voltage processes present in both observable voltage V₁ and observable voltage V₂. There is evidence of a cardiac waveform, as indicated by a repetitive pulse or pulse train. There is also evidence of a burst of a noise-like signal, possibly from a muscle contraction. The magnitude of the signals may be due to the positioning of the sensors 16 observing the voltages. For example, sensors 16a and 16b may be providing the observable signals (measured voltage) shown in the upper graph of FIG. 4, while sensors 16a and 16c may be providing the observable signals (measured voltage) shown in the lower graph of FIG. 4. Both graphs, nonetheless, indicate the two distinct voltage processes.

Depending upon what measured voltage is desired to be isolated, the mesh equations can be manipulated to so isolate that measured voltage. For example, using Kirchoff's voltage law, the two mesh equations can be manipulated to isolate the measured voltage of the heart as
V_{H}=−(V₁/r₁)(r₁+r₃+r₄+r₆)+(V₂/r₂)r₄.

One method for isolating the heart signal V_{H} is to estimate the five resistances r₁, r₂, r₃, r₄, and r₆. This may be accomplished by iteratively adjusting each resistance so that an expected ECG waveform is produced. Alternatively, each resistance value may be assumed and subsequently adjusted so that the variance observed on what V_{H} shows as an ECG waveform is minimized. Further alternatively, a linear weighted sum of the observable voltages can be posited and weighting coefficients for nulling out the noise may be adjusted.

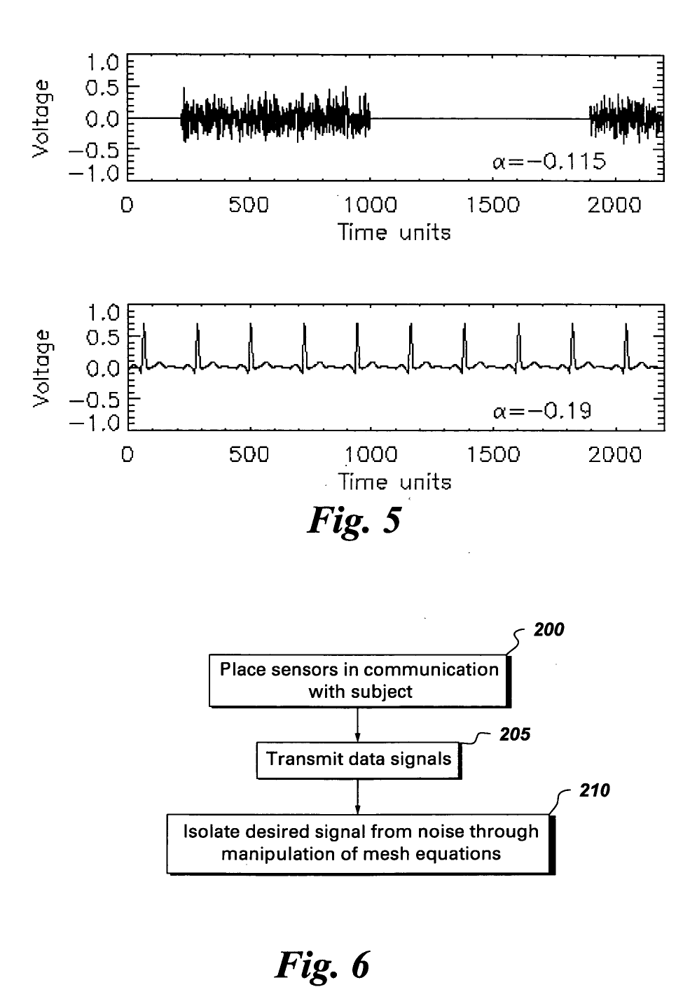

For the example depicted in FIG. 3, the resistances are chosen to be r₁=100, r₂=210, r₃=90, r₄=160, r₅=200, r₆=50, and r₇=90. The voltage signals V_{H} and V_{N} can be formed and plotted based upon the equation
V=−V₁−αV₂.
The value of α, which is a constant, is chosen to create isolation of one of the voltage signals from the other voltage signals. One manner in which the value of α may be chosen is through trial and error, while another manner is through iterative techniques. Utilizing an α of −0.115, the noise signal can be isolated, and choosing an α of −0.19 allows for isolation of the heart signal. The results of these choices are shown in FIG. 5. The isolated noise signal is depicted in the upper graph, and the isolated heart signal is depicted in the lower graph.

Referring now to FIG. 2, there is shown another embodiment of the invention. Instead of utilizing wired sensors, such as sensors 16, the physiological parameter monitoring system 110 of FIG. 2 includes a table 112 with a monitoring pad 114 positioned thereon. The monitoring pad 114 includes a plurality of sensor sites 116. The monitoring pad 114 may be formed of any material suitable for incorporating sensor sites 116 and having a relative degree of comfort.

Alternative to the embodiments illustrated in FIGS. 1 and 2, the sensors may be incorporated within a garment that may be worn by the subject. Just as the sensor sites 116 are dispersed through the monitoring pad 114, similarly sensors may be dispersed throughout the garment and configured to communicate with the data manager. The garment may include a communication line connectable with the data manager, or instead, the sensors may be configured to wirelessly communicate with the data manager.

The sensor sites 116 obtain physiological data, such as observable voltage signals. The physiological data obtained by the sensor sites 116 is then transmitted to a computational device 118, which includes a data manager 120 and a data auxiliary device 122. As illustrated, the physiological data is wirelessly transmitted to the computational device 118, although it should be appreciated that any method of transmitting the data, wirelessly or wired, may be utilized. The data manager 120 is configured to assemble and format the physiological data from the sensor sites 116. Specifically, the data manager 120 manipulates the linear combinations of the voltage differences between the electrode pairs so that the sought after observed voltage (such as, for example, the heartbeat) is rendered as clean of artifacts as possible. The manipulation may be accomplished in several ways. For example, the α, or a set of α, may be adjusted by hand by a technician viewing the output and enhancing the quality of the output. Further, the manipulation may be accomplished through the utilization of a variety of artificial intelligence algorithms that seek to enhance the likelihood of a clean prescribed model signal. The data auxiliary device 122 is configured to receive the assembled and formatted data from the data manager 120.

Next, and with specific reference to FIG. 6, will be described a method for isolating an electrical signal containing physiological data of a subject. At Step 200, sensors are placed in communication with the subject. The sensors may be sensors 16 (FIG. 1), or they may be sensor sites 116 (FIG. 2). At Step 205, physiological data is obtained by the sensors and transmitted to a computational device. The transmission of the physiological data may be wireless or wired transmission. Finally, at Step 210, the desired signal, such as a heart signal, is isolated from other signals found within the physiological data transmitted to the computational device. The isolation is accomplished by manipulating mesh equations that model the physiological data of at least two of the sensors. This may be accomplished through the use of resistive circuit analytical techniques, such as Kirchoff's voltage law.

An added benefit of the described technique is that it allows identification of a disconnected or unconnected electrode. Specifically, voltage potentials sensed with respect to a disconnected electrode will exhibit no variation. Such a condition may be easily recognized by an attendant or a computer-implemented algorithm.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed as new and desired to be protected by Letters Patent of the United States is:

1. A physiological parameter monitoring system, comprising:
a plurality of sensors configured to obtain from a subject at least one observable voltage containing two or more signals;
a data manager in communication with said plurality of sensors and being configured to assemble and format data obtained by said plurality of sensors; and
a data auxiliary device configured to receive the assembled and formatted data from said data manager;
wherein said data manager is configured to isolate one of said two or more signals.

2. The system of claim 1, wherein said at least one observable voltage is a potential difference between two of said plurality of sensors.

3. The system of claim 1, wherein said data manager isolates said one of said two or more signals by manipulating mesh equations that relate to measured voltages of at least two of said plurality of sensors.

4. The system of claim 3, wherein the manipulating of mesh equations is accomplished through the use of resistive circuit analytical techniques.

5. The system of claim 4, wherein the resistive circuit analytical techniques comprise Kirchoff's voltage law.

6. The system of claim 4, comprising means for identifying a non-varying signal.

7. The system of claim 1, further comprising a table upon which the subject is positionable.

8. The system of claim 7, wherein said plurality of sensors is embedded within a pad positionable upon said table.

9. The system of claim 1, wherein said plurality of sensors is on a garment to be worn by the subject.

10. The system of claim 1, wherein said plurality of sensors wirelessly communicates with said data manager.

11. The system of claim 1, wherein said plurality of sensors comprises one or more sensors from the group consisting of capacitive sensors, single electrode sensors, and Laplacian electrode sensors.

12. The system of claim 1, wherein the signal to be isolated comprises one from the group consisting of an electrocardiogram (ECG) of the subject, an electroencephalogram (EEG) of the subject, a heart rate of the subject, an ECG of a fetus within the subject, and a heart rate of a fetus within the subject.

13. A physiological parameter monitoring system, comprising:
a plurality of sensors configured to obtain from a subject at least one observable voltage containing two or more signals, said at least one observable voltage being a potential difference between two of said plurality of sensors;
a data manager in communication with said plurality of sensors and being configured to assemble and format data obtained by said plurality of sensors; and
a data auxiliary device configured to receive the assembled and formatted data from said data manager;
wherein said data manager is configured to isolate one of said two or more signals by manipulating mesh equations that relate to measured voltages of said two of said plurality of sensors.

14. The system of claim 13, wherein the manipulating of mesh equations is accomplished through the use of resistive circuit analytical techniques.

15. The system of claim 13, further comprising a table upon which the subject is positionable.

16. The system of claim 15, wherein said plurality of sensors is on a pad positionable upon said table.

17. The system of claim 13, wherein said plurality of sensors is on a garment to be worn by the subject.

18. The system of claim 13, wherein said plurality of sensors wirelessly communicates with said data manager.

19. The system of claim 13, wherein said plurality of sensors comprises one or more sensors from the group consisting of capacitive sensors, single electrode sensors, and Laplacian electrode sensors.

20. The system of claim 13, wherein the signal to be isolated comprises one from the group consisting of an electrocardiogram (ECG) of the subject, an electroencephalogram (EEG) of the subject, a heart rate of the subject, an ECG of a fetus within the subject, and a heart rate of a fetus within the subject.

21. A method for collecting and monitoring physiological parameters, comprising:
placing a plurality of sensors in communication with a subject, said plurality of sensors being configured to obtain a plurality of voltage signals from the subject;
transmitting data from said plurality of sensors to a data manager; and
isolating a desired voltage signal from the plurality of voltage signals, wherein the data manager isolates the desired voltage signal through a manipulation of mesh equations that relate to measured voltages of at least two of said plurality of sensors.

22. The method of claim 21, wherein each of said plurality of voltage signals is a potential difference between two of said plurality of sensors.

23. The method of claim 21, wherein said manipulation of mesh equations is accomplished through the use of resistive circuit analytical techniques.

24. The method of claim 21, wherein said plurality of sensors is on a pad positionable upon said table.

25. The method of claim 21, wherein said plurality of sensors is on a garment to be worn by the subject.

26. The method of claim 21, wherein said transmitting data is accomplished wirelessly.

27. The method of claim 21, wherein said plurality of sensors comprises one or more sensors from the group consisting of capacitive sensors, single electrode sensors, and Laplacian electrode sensors.

28. The method of claim 21, wherein the desired voltage signal to be isolated comprises one from the group consisting of an electrocardiogram (ECG) of the subject, an electroencephalogram (EEG) of the subject, a heart rate of the subject, an ECG of a fetus within the subject, and a heart rate of a fetus within the subject.

29. A method for collecting and monitoring physiological parameters, comprising:
placing a plurality of sensors in communication with a subject, said plurality of sensors being configured to obtain a plurality of voltage signals from the subject, wherein each of said plurality of voltage signals is a potential difference between two of said plurality of sensors;
transmitting data from said plurality of sensors to a data manager; and
isolating a desired voltage signal from the plurality of voltage signals, wherein the data manager isolates the desired voltage signal through a manipulation of mesh equations that relate to measured voltages of at least two of said plurality of sensors, wherein said manipulation of mesh equations is accomplished through the use of resistive circuit analytical techniques.

30. The method of claim 29, wherein the resistive circuit analytical techniques comprise Kirchoff's voltage law.

31. The method of claim 29, wherein said plurality of sensors is on a pad positionable upon said table.

32. The method of claim 29, wherein said plurality of sensors is on a garment to be worn by the subject.

33. The method of claim 29, wherein said transmitting data is accomplished wirelessly.

34. The method of claim 29, wherein said plurality of sensors comprises one or more sensors from the group consisting of capacitive sensors, single electrode sensors, and Laplacian electrode sensors.

35. The method of claim 29, wherein the desired voltage signal to be isolated comprises one from the group consisting of an electrocardiogram (ECG) of the subject, an electroencephalogram (EEG) of the subject, a heart rate of the subject, an ECG of a fetus within the subject, and a heart rate of a fetus within the subject.
